(19) United States
(12) Patent Application Publication
(10) Pub. No.: US 2014/0309511 A1
(43) Pub. Date: Oct. 16, 2014

(54) Medical arrangements and a method for prediction of a value related to a medical condition

(71) Applicant: DIANOVATOR AB, Linhamn (SE)

(72) Inventor: Fredrik Stål, Limhamn (SE)

(21) Appl. No.: 14/362,918

(22) PCT Filed: Dec. 6, 2012
(86) PCT No.: PCT/SE2012/051348

§ 371 Date: Jun. 5, 2014

(30) Foreign Application Priority Data
Dec. 6, 2011 (SE) 1151161-5

(51) Int. Cl. A61B 5/00; A61M 15/00; A61B 5/11; A61M 37/00; A61B 5/145; A61M 5/142; A61M 15/08

(52) U.S. Cl. CPC A61B 5/7275; A61B 5/7225; A61B 5/7267; A61B 5/4839; A61M 5/14276; A61M 15/009; A61M 15/08; A61M 37/00; A61B 5/14532; A61B 5/4866; A61B 5/1118
USPC 600/365; 600/300; 600/595

(57) ABSTRACT

The disclosure is related to a medical device, a system, a method and a storage medium for prediction of a value related to a medical condition. More particularly the invention relates to prediction of glucose in the blood or prediction of blood pressure. The disclosure enables improved control of glucose in the blood or of blood pressure, since prediction can be made with higher accuracy, even when switching between dynamic modes, corresponding to different states, such as exercising. In one embodiment a medical device (1) is provided, which comprises: a predicting unit (2) for prediction of a value related to a medical condition of a patient at a future point in time, based on at least a measured present value related to the medical condition of the patient; wherein the predicting unit (2) comprises an ensemble predictor (3), for predicting the value at a future point in time, continuously adaptable to different predictor modes based on different states of the patient.

### BACKGROUND OF THE INVENTION

1. Field of the Invention

This disclosure pertains in general to the field of treatment of a medical condition. More particularly the disclosure relates to control of glucose in the blood or control of blood pressure. Even more particularly, the disclosure relates to prediction of a value related to a medical condition of a patient.

2. Description of the Prior Art

Diabetes is a medical condition, which may be difficult to remedy or treat. In order to treat or mitigate diabetes, a patient is given insulin. However, it is difficult to determine the amount of insulin, which should be administered to the patient, with good precision. In order to determine the amount of insulin to administer, the level of glucose in the blood needs to be predicted. Thus, a good method of predicting the level of glucose in the blood is needed. Similar predictions are needed in order to treat or mitigate a non-optimal blood pressure.

Different methods for predicting a glucose level in the blood or for predicting the blood pressure are known. These methods perform well for specific conditions. As an example, one method of predicting the glucose level in the blood of a patient may perform well when the patient is exercising but not so well in other situations, whereas another method may perform well while the patient is resting, but not so well in other situations.

From WO2005/081171 A2, an insulin bolus calculator for a mobile communication device is known. This document discloses a calculating device for indicating an amount of ets to be consumed by a patient, wherein the calculations are based on parameters. However, the advice to the patient is based on static calculations.

Thus, there may be a need for dynamic predictions.

There may also be a need for more accurate predictions.

Corresponding disadvantages may be found in other medical conditions, such as non-optimal blood pressure.

Thus, there may be a need for an improved method or system for making predictions for medical conditions, such as diabetes or non-optimal blood pressure.

### SUMMARY OF THE INVENTION

Accordingly, embodiments of the present disclosure preferably seek to mitigate, alleviate or eliminate one or more deficiencies, disadvantages or issues in the art, such as the above-identified, singly or in any combination by providing a medical device, a system, a computer-implemented method and a non-transitory computer-readable storage medium that provides prediction of a value related to a medical condition, according to the appended patent claims.

According to one aspect of the disclosure, a medical device is provided, which comprises a predicting unit for prediction of at least one value related to a medical condition of a patient at a future point in time, based on at least a measured present value related to the medical condition of the patient. The predicting unit comprises an ensemble predictor, for predicting the at least one value at a future point in time. The ensemble predictor is continuously adaptable to different predictor modes, e.g. based on different states of the patient. The result of the prediction, i.e. the predicted future value or values, may be used directly by the patient to control the medical condition. As an alternative, the result may be used by a physician, an advisor or an advising system for advising the patient how to control the medical condition. As another alternative, the prediction is used in a closed loop with a suitable controller for controlling the medical condition.

According to another aspect of the disclosure, a system for treating a medical condition, such as a non-optimal proportion of glucose in the blood or non-optimal blood pressure, is provided. The system comprises a measuring unit, for measuring a present value related to a medical condition of a patient. It also comprises a predicting unit for prediction of at least one value related to the medical condition of the patient at a future point in time, based on at least the measured present value. The system further comprises a calculating unit for calculating an amount of a substance, such as insulin or epinephrine, based on at least the predicted value at a future point in time. Also comprised is an administering unit for administer the amount of the substance to a patient at the future point in time in order to treat the medical condition.

According to yet another aspect of the disclosure, a computer implemented method for prediction of at least one value related to a medical condition of a patient at a future point in time, based on at least a measured present value related to the medical condition of the patient is provided. The method comprises predicting the value at a future point in time in an ensemble predictor of the predicting unit. The predicting is continuously adaptable to different predictor modes, e.g. based on different states of the patient.

According to a further aspect of the disclosure, a non-transitory computer-readable storage medium encoded with programming instructions is provided, wherein the storage medium is loaded into a computerized control system of a medical device, and the programming instructions cause the computerized control unit to control a prediction unit of the medical device during operation. This is performed by predicting, in an ensemble predictor of the predicting unit, at least one value related to a medical condition of a patient at a future point in time, e.g. based on at least a measured present value related to the medical condition of the patient. The ensemble predictor is continuously adapting to different predictor modes based on different states of the patient.

Further embodiments of the disclosure are defined in the dependent claims, wherein features for the second and subsequent aspects of the disclosure are as for the first aspect mutatis mutandis.

Some embodiments of the disclosure provide for that a more reliable and more accurate prediction can be performed. Prediction can be made with high accuracy, even when switching between dynamic modes, e.g. corresponding to different states, such as resting, sitting, walking, exercising, of a patient, occurs.

Some embodiments of the disclosure also provide for optimization of flexibility versus robustness of the predicting unit.

Some embodiments of the disclosure also provide for optimization of the dynamics of the system.

Some embodiments of the disclosure also provide for small predictive errors and/or sufficient time margins for alarms to be raised.

Some embodiments of the disclosure also provide for a margin to the borders of the normoglycemic region. Thus, the risk of leaving the normoglycemic region is reduced.

Some embodiments of the disclosure also provide for that the system will work satisfactory, even if there is a sensor failure or a loss of confidence in the estimated predictor mode.

Some embodiments of the disclosure also provide for easy initialization and fast adaptation to the current conditions.

Some embodiments of the disclosure also provide for optimized control of a medical condition.

Some embodiments of the disclosure also provide for a simplified control of and/or simplifying controlling medical conditions, such as diabetes and/or non-optimal blood pressure.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the disclosure are capable of will be apparent and elucidated from the following description of embodiments of the present disclosure, reference being made to the accompanying drawings, in which

FIG. 1 illustrates the core components of a medical device;

FIG. 2 illustrates the core components of a predicting unit;

FIG. 3 illustrates the core components of a system for treating a medical condition; and

FIG. 4 illustrates different steps of a computer implemented method for prediction of a value related to a medical condition of a patient.

### DESCRIPTION OF THE PREFERRED EMBODIMENTS

Specific embodiments of the disclosure will now be described with reference to the accompanying drawings. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. The terminology used in the detailed description of the embodiments illustrated in the accompanying drawings is not intended to be limiting of the disclosure. In the drawings, like numbers refer to like elements.

The following description focuses on an embodiment of the present disclosure applicable to prediction of a medical condition and in particular to prediction of an amount of glucose in the blood. However, it will be appreciated that the disclosure is not limited to this application but may be applied to many other medical conditions, including for example non-optimal blood pressure. Below the prediction is described as prediction of a value at a future point in time. However, in some embodiments, it is also possible to predict a plurality of values for different horizons, i.e. the prediction may be a prediction of values at multiple future points in time. Thus, a more versatile prediction may be performed.

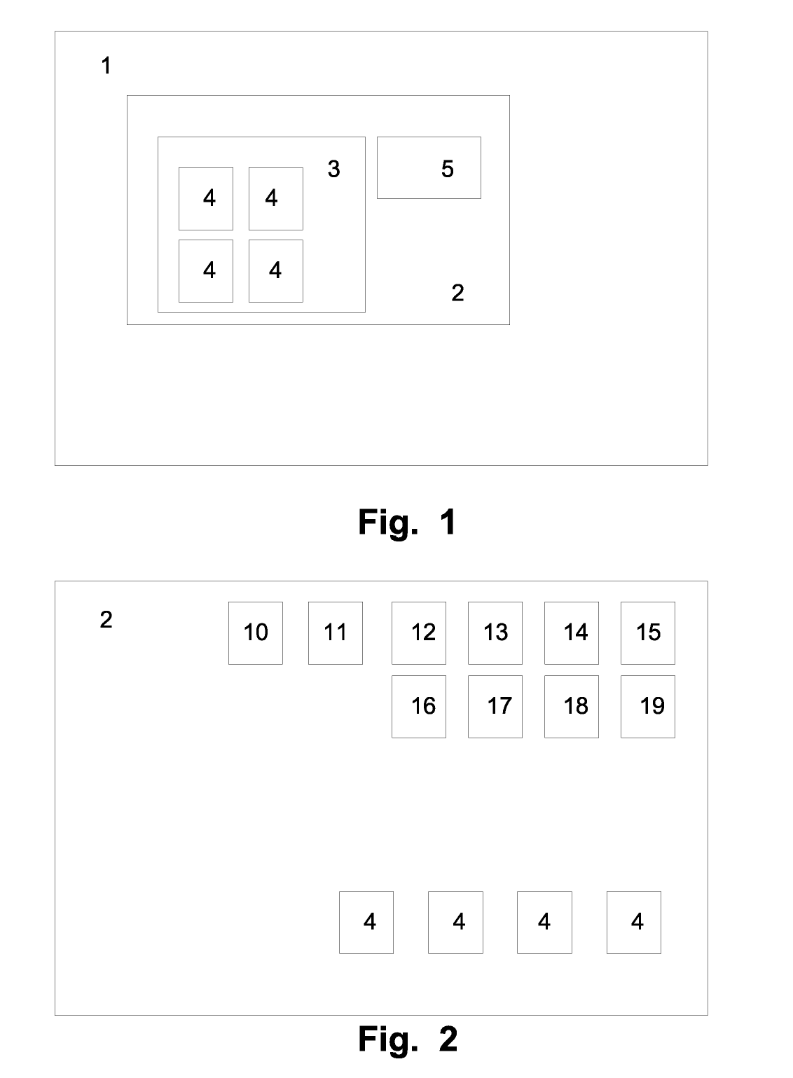

FIG. 1 shows the core components of a medical device 1. The medical device 1 comprises a predicting unit 2. The predicting unit 2 predicts a value related to a medical condition of a patient at a future point in time, based on at least a measured present value related to the medical condition of the patient. The prediction may also be based on previous measured values. The medical condition is for example diabetes and the measured value is then the amount of glucose in a patient's blood. This value may be measured invasively or non-invasively with an appropriate sensor. As an example, if measured non-invasively, a near infrared absorbance spectrum of in vivo skin tissue can be analyzed. Other optical methods or capacitance measurements may be used for non-invasive measuring. As another example, the current electrical capacitance of the outer surface of the skin can be measured and thereafter compared to stored data in order to determine a blood glucose level. As an alternative, the glucose may be measured in more than one way, e.g. invasively and non-invasively. Then, in order to produce one single measured glucose value, an average or possibly a weighted linear average of the different measurements may be calculated. As another alternative, the prediction may in addition be based on other measurements, such as a measured heart rate, a measured temperature, a value from an accelerometer or manual input from the user. The user may for instance input that he/she is entering a region with different climate. The predicting unit 2 comprises an ensemble predictor 3, for predicting the value at a future point in time. The ensemble predictor 3 is continuously adaptable to different predictor modes based on different states of the patient. The predicting unit also comprises a plurality of predictor units 4. There may be any number of predictor units 4. The predictor units 4 receive at least the measured present value as an input. The predictor units 4 may in addition receive previous measured values as input. Furthermore, the predictor units 4 may also receive information about food intake, insulin intake, a physical activity level, exercise and/or other user provided information as input. The predictor units 4 are each assigned a weight. Thus, the output of the predictor units 4 can be weighted. From the weighted output of each of the predictor units 4, the ensemble predictor 3 can be obtained by the use of sliding window Bayesian model averaging. Bayesian model averaging is an ensemble technique that seeks to approximate the Bayes Optimal Classifier by sampling hypotheses from the hypothesis space, and combining them using Bayes' law. With the ensemble predictor 3, a more reliable and more accurate prediction can be provided. Thus, prediction can be made with high accuracy. One advantage of the ensemble predictor 3 is that it performs well, even when switching between dynamic modes occurs. Such dynamic modes may correspond to different situations or states of the patient, such as resting, sitting, walking, exercising etc. A switch from one dynamic mode to another dynamic mode may instead or in addition relate to other changes in circumstances or other changes in parameters. In FIG. 1, a regularization unit 5 can also be seen. This unit is adapted for optimizing the relation between flexibility and a robustness of the predicting unit 2. If the flexibility of the predicting unit 2 is increased, the robustness of it normally decreases and vice versa. Thus, there may be a need for a trade-off or optimization between flexibility and robustness in order to provide an optimal prediction. Furthermore, if previous measured values are used in order to calculate a value related to a medical condition of a patient at a future point in time, the calculations may include a forgetting factor in order to optimize the dynamics of the predicting unit. This forgetting factor is chosen so that there is a good balance between agility towards transients or disturbances and sensitivity to noise. The prediction unit utilizes a cost function for determining the weights. The cost function may be the 2-norm, which is a natural choice. However, in some embodiments an asymmetric cost function may be utilized, so that the prediction error cost increases with the absolute glucose value and/or the sign of the prediction error. The use of an asymmetric cost function secures or at least increases the probability of keeping the prediction inside a certain zone, such as zone A of the Clarke Grid Error Plot, which may be more difficult to do with utilization of just the 2-norm. Thus, it may be safer to utilize an at least partly asymmetric cost function. Thereby, small predictive errors and sufficient time margins for alarms to be raised, may be provided. Alternatively, other norms, such as the Manhattan norm may be utilized.

In one embodiment, a nominal mode is utilized for initialization of the predicting unit 2. In this mode, all predictor units 4 have equal weights. This mode may also be utilized as a fallback mode, which may be utilized during sensor failure or other unpredictable behavior. By the use of this mode, the predicting unit will be initialized easily and will quickly adapt to the current conditions. The predicting unit may also perform well, even if there is a sensor failure or a loss of confidence in the estimated predictor mode. However, as an alternative of using a fallback mode, the predictor may continue at the present predictor mode during sensor failure or other unpredictable behavior.

According to an embodiment described below with reference to FIG. 2, the predicting unit 2 comprises different modules. The predicting unit 2 comprises a predictor storage module 10 for storing a plurality of predictors 4. The predictors 4 can be determined beforehand. Thus, the predictors 4 can be predetermined. However, it is still possible to update predictors 4. This is typically done when new prediction algorithms are available. The prediction algorithms are in one embodiment updated, while the use of the predicting unit, the medical device or the system is paused. The predicting unit 2 further comprises a database 11, containing training data, which training data has been obtained and thereafter stored in the database. The predicting unit 2 also comprises a processing module 12 for running a constrained estimation formula. The constrained estimation formula may be:

The formula is run on training data, k is the time instance and T_{P}_{ᵢ} represents the time points corresponding to a dynamic mode Pᵢ, N is the size of the evaluation window, wₖ is an array of weights, ŷ₁ is an array of predictor units and L(yⱼ,ŷⱼ) is a cost function. The predicting unit 2 further comprises a weight retrieving module 13 for retrieving the sequence of weights given the dynamic mode according to {w_{k|P}_{ᵢ}}T_{P}_{ᵢ}, ∀iε{1, . . . , n}.
The prediction unit 2 also comprises a classification module 14 for classifying different dynamic modes. It further comprises a probability density function determination module 15 for determining probability density functions p(wₖ|pᵢ) for each dynamic mode from training results by supervised learning. A supervised learning algorithm analyzes the training data and produces an inferred function, which is called a classifier if the output is discrete or a regression function if the output is continuous. The inferred function should predict the correct output value for any valid input object. This requires the learning algorithm to generalize from the training data to unseen situations in a “reasonable” way. The predicting unit 2 also comprises a probability estimator 16, which if possible, estimates a probability for a certain dynamic mode given the data, i.e. p(P|D). Information about dynamic modes may be an input to the predicting unit 2. Such information may be retrieved from an additional sensor. The information may also be a result of correlation calculations from e.g. sensor signals and data for predictor modes. Such a priori information may further improve the prediction of future values. If it is not possible to estimate a probability for a certain dynamic mode given the data, this probability is assumed to be equal for all the different dynamic modes and can therefore be disregarded. The probability estimator 16 estimates the probability for weights given a certain dynamic mode, i.e. p(wₖ|pₖ). Also an initializer 17 for initializing by setting a present predictor mode to a nominal mode is comprised by the predicting unit 2. The predicting unit 2 further comprises a calculation unit 18 for calculating an array of weights wₖ for each time step and for a present dynamic mode according to:

$${w}_{k \vert {p}_{k - 1}} = arg min {\sum }_{j = k - N}^{k - 1} {μ}_{j}^{k - j} ℒ ( {y}_{j} , {w}_{k \vert {p}_{k - 1}}^{T} {\hat{y}}_{j} ) + ( {w}_{k \vert {p}_{k - 1}} - {w}_{0 \vert {p}_{k - 1}} ) {R ( {w}_{k \vert {p}_{k - 1}} - {w}_{0 \vert {p}_{k - 1}} )}^{T}$$

$$s . t .1 {w}_{k \vert Pk - 1} = 1. ,$$

wherein μⱼ is a forgetting factor and R is a regularization matrix. The predicting unit also comprises a mode switcher 19 for determining if switching to another predictor mode should be performed, according to:

$$if for any i \neq {p}_{k - 1} \text{:}$$

$$\{ \begin{matrix}p ( {P}_{i} \vert {w}_{k \vert {p}_{k - 1}} , {D}_{k} ) > λ , and \\ p ( {w}_{k \vert {p}_{k - 1}} \vert {P}_{i} , {D}_{k} ) > δ\end{matrix} \\ where \\ p ( {P}_{i} \vert {w}_{k \vert {p}_{k - 1}} , {D}_{k} ) = \frac{p ( {w}_{k \vert {p}_{k - 1}} \vert {P}_{i} , {D}_{k} ) p ( {P}_{i} \vert {D}_{k} )}{{\sum }_{j} p ( {w}_{k \vert {p}_{k - 1}} \vert {P}_{j} , {D}_{k} ) p ( {P}_{j} \vert {D}_{k} )} ,$$

wherein λ and δ are constants and D is data. If it is possible to estimate probabilities for the predictor modes a priori, then these are accounted for in the equation. Such estimation of probabilities for the predictor modes may utilize sensor signals, information about food intake, insulin intake, a physical activity level, exercise and/or other user provided information as input. Otherwise, these probabilities are assumed equal, and do not have to be accounted for in the above equation. If the mode switcher 19 determines that switching to another predictor mode should be performed, it also triggers the calculation unit 18 to recalculate the array of weights wₖ. By the use of the above specified modules, predictions for a medical condition may be optimized and thus enable an accurate control of the medical condition.

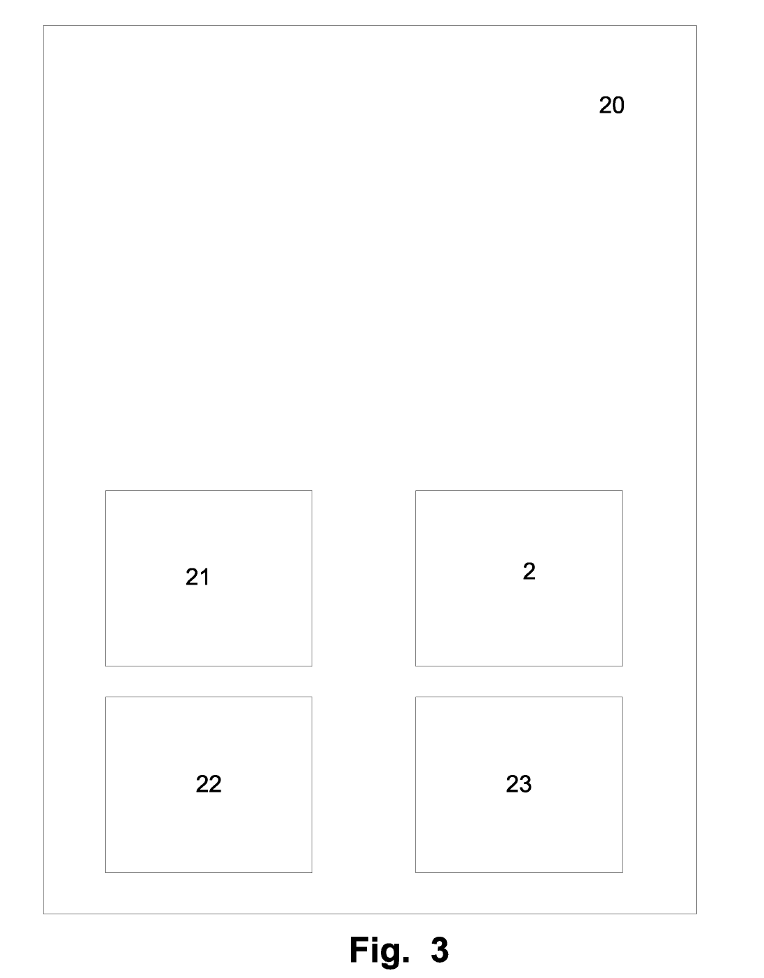

A further embodiment of the disclosure is illustrated in FIG. 3. In FIG. 3, a system 20 for treating a medical condition, such as a non-optimal proportion of glucose in the blood or non-optimal blood pressure, can be seen. The system comprises a measuring unit 21, for measuring a present value related to a medical condition of a patient. It also comprises a predicting unit 2 for prediction of a value related to the medical condition of the patient at a future point in time, based on at least the measured present value. It further comprises a calculating unit 22 for calculating an amount of a substance, based on at least the predicted value at a future point in time. The substance may be a hormone, such as insulin or epinephrine or another substance, depending on the medical condition. The system 20 also comprises an administering unit 23 for administering the amount of the substance to a patient at the future point in time in order to treat the medical condition. The administering unit may e.g. be a subcutaneous or implantable electronic infusion pump, an insulin pen, a nose spray or a patch to put on the skin. As an alternative, a plurality of administering units may be used. The measuring unit is in one embodiment a continuous glucose measurement system. The system simplifies the control of medical conditions, such as diabetes and/or non-optimal blood pressure.

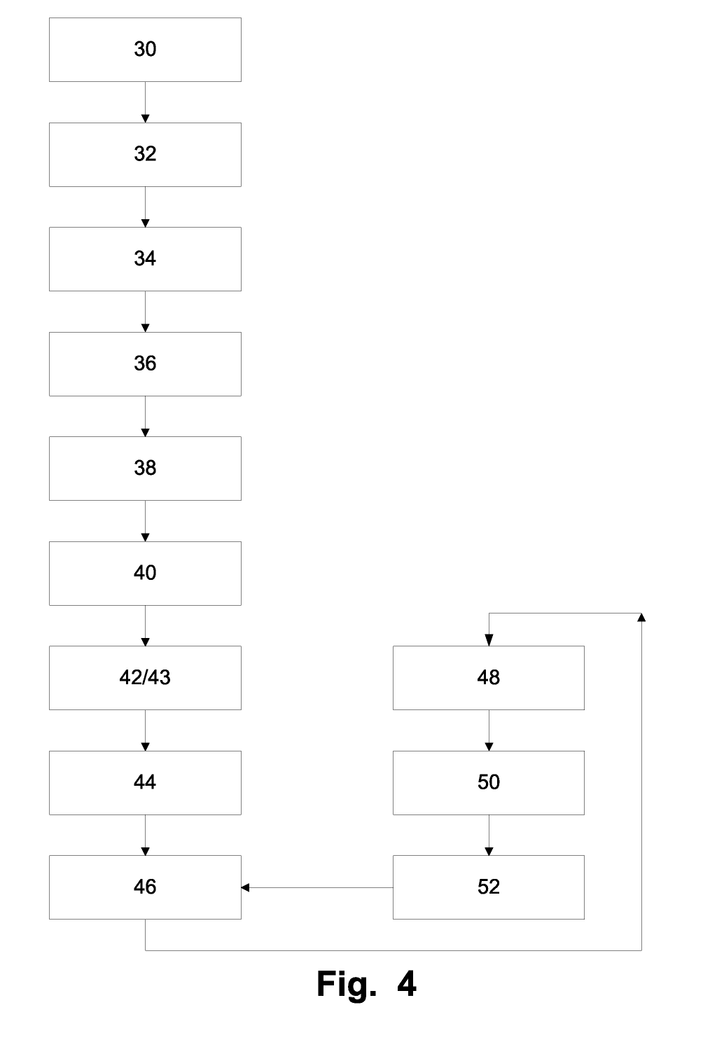

A computer implemented method for prediction of a value related to a medical condition of a patient at a future point in time, based on at least a measured present value related to the medical condition of the patient is also disclosed. This method comprises predicting the value at a future point in time in an ensemble predictor 3 of the predicting unit 2. The prediction is continuously adaptable to different predictor modes, which are e.g. based on different states of the patient. Such states may be resting, sitting, walking, exercising etc. With reference to FIG. 4, the computer implemented method may further comprise estimation 30 of a predictor for each of a plurality of predictor units 4. Furthermore, the method comprises obtainment 32 of training data and storing of the training data in a database. It also comprises execution 34 of the constrained estimation:

$$\{ {w}_{k \vert {P}_{i}} \} {T}_{{P}_{i}} = arg min {\sum }_{i = k - N / 2}^{k + N / 2} ℒ ( y ( {t}_{i} ) , {w}_{k \vert {P}_{i}} {}_{}^{T}{\hat{y}}{}_{i}^{} ) , \\ k ∈ {T}_{{P}_{i}}$$

$$s . t . 1 {w}_{k \vert {P}_{i}} = 1.$$

on training data, where T_{P}_{ᵢ} represents the time points corresponding to a dynamic mode Pᵢ, N is the size of the evaluation window, wₖ is an array of weights, ŷ₁ is an array of predictor units and L(yⱼ,ŷⱼ) is a cost function. Thereby training results are obtained. Furthermore, it also comprises retrieving 36 the sequence of {w_{k|P}_{ᵢ}}T_{P}_{ᵢ}, ∀iε{1, . . . , n} The method further comprises classification 38 of different predictor modes. The centers of the different predictor modes w_{0|P}_{ᵢ}=E(w|Pᵢ) are estimated from training data. The method also comprises determination 40 of probability density functions p(w_{k|P}_{ᵢ}) for each predictor mode from training results by supervised learning. When the centers and the probability density functions of each predictor mode have been determined, these determined values are used for all predictions. However, in one embodiment, the predictor modes are updated in a recursive manner. This may be beneficial, since not just recorded training data, but also all new data obtained during use, can be used for estimating predictor modes. When predictor modes are updated in a recursive manner, a forgetting factor can be used. Thus, the predictor modes can adapt to new data. The method also comprises estimation 42 of a probability for a certain dynamic mode given data, i.e. p(P|D), if possible. Otherwise the probability for weights given a certain predictor mode, i.e. p(wₖ|pₖ), is estimated and utilized 43. In the method, initialization 44 is performed, by putting a present predictor mode to a nominal mode. Furthermore, the method comprises a step 46 for calculating the array of weights wₖ for each time step and for a present predictor mode as:

$${w}_{k \vert {p}_{k - 1}} = arg min {\sum }_{j = k - N}^{k - 1} {μ}_{j}^{k - j} ℒ ( {y}_{j} , {w}_{k \vert {p}_{k - 1}}^{T} {\hat{y}}_{j} ) + ( {w}_{k \vert {p}_{k - 1}} - {w}_{0 \vert {p}_{k - 1}} ) {R ( {w}_{k \vert {p}_{k - 1}} - {w}_{0 \vert {p}_{k - 1}} )}^{T}$$

$$s . t . 1 {w}_{k \vert {p}_{k - 1}} = 1. ,$$

wherein μⱼ is a forgetting factor and R is a regularization matrix. The method also comprises determining in a step 48 if switching to another predictor mode should be performed, according to:

$$if for any i \neq {p}_{k - 1} \text{:}$$

$$\{ \begin{matrix}p ( {P}_{i} \vert {w}_{k \vert {p}_{k - 1}} , {D}_{k} ) > λ , \\ p ( {w}_{k \vert {p}_{k - 1}} \vert {P}_{i} , {D}_{k} ) > δ\end{matrix} and \\ where \\ p ( {P}_{i} \vert {w}_{k \vert {p}_{k - 1}} , {D}_{k} ) = \frac{p ( {w}_{k \vert {p}_{k - 1}} \vert {P}_{i} , {D}_{k} ) p ( {P}_{i} \vert {D}_{k} )}{{\sum }_{j} p ( {w}_{k \vert {p}_{k - 1}} \vert {P}_{j} , {D}_{k} ) p ( {P}_{j} \vert {D}_{k} )} ,$$

wherein λ and δ are constants and D is data.

In step 50, it is determined if prediction should be continued. If prediction should be continued then steps 46-50 are repeated. This repetition 52 is performed until it is determined that the prediction should not be continued any longer.

In one embodiment, there is a non-transitory computer-readable storage medium encoded with programming instructions. The storage medium is loaded into a computerized control system of a medical device, and the programming instructions cause the computerized control unit to control a prediction unit 2 of the medical device during operation. This control comprises predicting, in an ensemble predictor 3 of the predicting unit 2, a value related to a medical condition of a patient at a future point in time, based on at least a measured present value related to the medical condition of the patient. The control also comprises continuously adapting to different predictor modes based on different states of the patient.

The present disclosure has been described above with reference to specific embodiments. However, other embodiments than the above described are equally possible within the scope of the disclosure. Different method steps than those described above, may be provided within the scope of the disclosure. The different features and steps of the disclosure may be combined in other combinations than those described. The scope of the disclosure is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications, for which the teachings of the present disclosure is/are used.

1. A medical device comprising:
a predicting unit for predicting a value related to a medical condition of a patient at a future point in time based on a measured present value related to said medical condition of said patient;
wherein said predicting unit comprises a plurality of predictor units and an ensemble predictor obtained from a weighted output of each of said predictor units for predicting said value at a future point in time continuously adaptable to different predictor modes.

2. The medical device of claim 1, wherein said ensemble predictor is configured for continuously adapting to different predictor modes based on different states of said patient.

3. The medical device of claim 1, wherein said ensemble predictor is obtained from sliding window Bayesian model averaging.

4. The medical device of claim 3, wherein said predicting unit further comprises a regularization unit for optimizing a flexibility and a robustness of said predicting unit.

5. The medical device of claim 3, wherein a forgetting factor is adapted in said predicting unit for optimizing the dynamics of said predicting unit.

6. The medical device of claim 3, wherein said predicting unit is configured for utilizing a cost function for determining said weights, and wherein said cost function is a 2-norm or an asymmetric cost function.

7. The medical device of claim 3, wherein a nominal mode, having equal weights for all predictor units, is utilized for initialization and/or as a fallback mode, said fallback mode being utilized during sensor failure or other unpredictable behavior.

8. The medical device of claim 3, wherein said predicting unit further comprises:
a) a predictor storage module for storing a predictor for each of said plurality of predictor units;
b) a database containing training data;
c) a processing module configured for running a constrained estimation formula,
$$\{ {w}_{k \vert {P}_{i}} \} {T}_{{P}_{i}} = arg min {\sum }_{i = k - N / 2}^{k + N / 2} ℒ ( y ( {t}_{i} ) , {w}_{k \vert {P}_{i}} {}_{}^{T}{\hat{y}}{}_{i}^{} ) , \\ k ∈ {T}_{{P}_{i}}$$
$$s . t . 1 {w}_{k \vert {P}_{i}} = 1. [ [ , ] ]$$
on training data, wherein T_{P}_{ᵢ} represents time points corresponding to a dynamic mode Pᵢ, N is the size of the evaluation window, wₖ is an array of weights, ŷᵢ is an array of predictor units and L(yⱼ,ŷⱼ) is a cost function;
d) a weight retrieving module configured for retrieving a sequence of weights given a predictor mode according to:
{w_{k|P}_{ᵢ}}T_{P}_{ᵢ}, •iε{1, . . . , n};
e) a classification module configured for classifying different predictor modes;
f) a probability density function determination module for determining probability density functions P (W_{k|Pi}) for each predictor mode from training results by supervised learning;
g) a probability estimator, which if possible estimates a probability for a certain dynamic mode given data p(P|D);
h) an initializer for initializing by setting a present predictor mode to a nominal mode;
j) a calculation unit configured for calculating an array of weights wₖ for each time step and for a present predictor mode according to
$${w}_{k \vert {p}_{k - 1}} = arg min {\sum }_{j = k - N}^{k - 1} {μ}_{j}^{k - j} ℒ ( {y}_{j} , {w}_{k \vert {p}_{k - 1}}^{T} {\hat{y}}_{j} ) + ( {w}_{k \vert {p}_{k - 1}} - {w}_{0 \vert {p}_{k - 1}} ) {R ( {w}_{k \vert {p}_{k - 1}} - {w}_{0 \vert {p}_{k - 1}} )}^{T}$$
$$s . t . 1 {w}_{k \vert {p}_{k - 1}} = 1. [ [ , ] ]$$
wherein μⱼ is a forgetting factor, R is a regularization matrix and present predictor mode center w_{0|p}_{_{k−1}}=E(w|pₖ₋₁); and
k) a mode switcher configured for determining if switching to another predictor mode should be performed, according to:
$$\underline{if} for any i \neq {p}_{k - 1} \text{:}$$
$$\{ \begin{matrix}p ( {P}_{i} \vert {w}_{k \vert P k - 1} , {D}_{k} ) > λ , \\ p ( {w}_{k \vert P k - 1} \vert {P}_{i} , {D}_{k} ) > δ\end{matrix} and \\ where \\ p ( {P}_{i} \vert {w}_{k \vert {p}_{k - 1}} , {D}_{k} ) = \frac{p ( {w}_{k \vert {p}_{k - 1}} \vert {P}_{i} , {D}_{k} ) p ( {P}_{i} \vert {D}_{k} )}{{\sum }_{j} p ( {w}_{k \vert {p}_{k - 1}} \vert {P}_{j} , {D}_{k} ) p ( {P}_{j} \vert {D}_{k} )} ,$$
wherein λ and δ are constants, and for triggering said calculation unit to recalculate said array of weights wₖ if it is determined that switching to another predictor mode should be performed.

9. The medical device of claim 8, wherein said probability estimator estimates a probability for a certain dynamic mode based on sensor signals, information about food intake, insulin intake, a physical activity level, exercise and/or other user provided information

10. A system for treating a medical condition, said system comprising:
a measuring unit, configured for measuring a present value related to a medical condition of a patient;
said predicting unit of claim 1;
a calculating unit configured for calculating an amount of a substance based on said predicted value at a future point in time; and
an administering unit configured for administering said amount of said substance to a patient at said future point in time in order to treat said medical condition.

11. The system of claim 10, wherein said administering unit is a subcutaneous or implantable electronic infusion pump, an insulin pen, a nose spray or a patch to put on the skin.

12. The system of claim 10, wherein said measuring unit is a continuous glucose measurement system, a non-invasive glucose measuring system, a glucose meter, or a combination thereof.

13. A computer implemented method for prediction of a value related to a medical condition of a patient at a future point in time based on a measured present value related to said medical condition of said patient, said method comprising
predicting said value at a future point in time in an ensemble predictor obtained from a weighted output of each of a plurality of predictor units of a predicting unit, wherein said predicting is continuously adaptable to different predictor modes.

14. The computer implemented method of claim 13, wherein said ensemble predictor is configured for continuously adapting to different predictor modes based on different states of said patient.

15. The computer implemented method of claim 13, further comprising:
a) storing predictors of a plurality of predictor units;
b) obtaining training data and storing said training data in a database;
c) running the constrained estimation:
$$\{ {w}_{k \vert {P}_{i}} \} {T}_{{P}_{i}} = arg min {\sum }_{i = k - N / 2}^{k + N / 2} ℒ ( y ( {t}_{i} ) , {w}_{k \vert {P}_{i}} {}_{}^{T}{\hat{y}}{}_{i}^{} ) , \\ k ∈ {T}_{{P}_{i}}$$
$$s . t . 1 {w}_{k \vert {P}_{i}} = 1.$$
on training data, wherein T_{P}_{ᵢ} represents the time points corresponding to a dynamic mode Pᵢ, N is the size of the evaluation window, wₖ is an array of weights, ŷ₁ is an array of predictor units and L(yⱼ,ŷ₁) is a cost function;
d) retrieving the sequence of {w_{k|P}_{ᵢ}}T_{P}_{ᵢ}, ∀iε{1, . . . , n};
e) classifying different predictor modes;
f) determining probability density functions p(w_{k|P}_{ᵢ}) for each predictor mode from training results by supervised learning
g) if possible estimating a probability for a certain dynamic mode given data p(P|D);
h) initializing, by putting a present predictor mode to a nominal mode;
j) calculating the array of weights wₖ for each time step and for a present predictor mode as:
$${w}_{k \vert {p}_{k - 1}} = arg min {\sum }_{j = k - N}^{k - 1} {μ}_{j}^{k - j} ℒ ( {y}_{j} , {w}_{k \vert {p}_{k - 1}}^{T} {\hat{y}}_{j} ) + ( {w}_{k \vert {p}_{k - 1}} - {w}_{0 \vert {p}_{k - 1}} ) {R ( {w}_{k \vert {p}_{k - 1}} - {w}_{0 \vert {p}_{k - 1}} )}^{T}$$
$$s . t . 1 {w}_{k \vert {p}_{k - 1}} = 1. ,$$
wherein μⱼ is a forgetting factor and R is a regularization matrix;
k) determining if switching to another predictor mode should be performed, according to:
If for any i≠pₖ₋₁:
$$\{ \begin{matrix}p ( {P}_{i} \vert {w}_{k \vert {p}_{k - 1}} , {D}_{k} ) > λ , \\ p ( {w}_{k \vert {p}_{k - 1}} \vert {P}_{i} , {D}_{k} ) > δ\end{matrix} and \\ where \\ p ( {P}_{i} \vert {w}_{k \vert {p}_{k - 1}} , {D}_{k} ) = \frac{p ( {w}_{k \vert {p}_{k - 1}} \vert {P}_{i} , {D}_{k} ) p ( {P}_{i} \vert {D}_{k} )}{{\sum }_{j} p ( {w}_{k \vert {p}_{k - 1}} \vert {P}_{j} , {D}_{k} ) p ( {P}_{j} \vert {D}_{k} )} ,$$
and
l) determining if prediction should be continued and if prediction should be continued then returning to step j.

16. The computer implemented method of claim 15, wherein said probability estimator estimates a probability for a certain dynamic mode based on sensor signals, information about food intake, insulin intake, a physical activity level, exercise and/or other user provided information.

17. A non-transitory computer-readable storage medium encoded with programming instructions, said storage medium being loaded into a computerized control system of a medical device, and said programming instructions causing said computerized control unit to control a prediction unit of the medical device during operation by:
predicting, in an ensemble predictor obtained from a weighted output of each of a plurality of predictor units of said predicting unit, a value related to a medical condition of a patient at a future point in time, based on a measured present value related to said medical condition of said patient and continuously adapting to different predictor modes.
